(19) United States
(12) Patent Application Publication
(10) Pub. No.: US 2026/0258486 A1
(43) Pub. Date: Sep. 3, 2026

(54) Epigenetic Analysis After Single Cell Interactions Using Microfluidics

(71) Applicant: Northeastern University, Boston, MA (US)

(72) Inventors: Tania KONRY, Boston, MA (US); Agustin DE GANZO, Allston, MA (US); Judene A. THOMAS, Randolph, MA (US); Christina SHARKEY, Brookline, MA (US)

(21) Appl. No.: 18/993,273

(22) PCT Filed: Aug. 30, 2023
(86) PCT No.: PCT/US2023/031576

§ 371 Date: Jan. 10, 2025

Related U.S. Application Data
(60) Provisional application No. 63/402,461, filed on Aug. 30, 2022.
(60) Provisional application No. 63/413,233, filed on Oct. 4, 2022.
(60) Provisional application No. 63/427,086, filed on Nov. 21, 2022.

(51) Int. Cl. C12Q 1/6869; C12Q 1/6806; G01N 15/14; G01N 15/1433

(52) U.S. Cl. CPC C12Q 1/6869; C12Q 1/6806; G01N 15/1433; G01N 15/1459

(57) ABSTRACT

Single-cell methods are provided for investigating DNA and RNA interactions with regulatory proteins and evaluating the cytotoxicity of immune cells towards target cells, such as tumor cells. The methods utilize droplet microfluidics and imaging-based technology to facilitate the isolation and analysis of protein-bound nucleic acids from individual immune cells or apoptotic tumor cells.

### BACKGROUND

Inside the nucleus, DNA is wrapped around histone proteins to form nucleosomes. Nucleosomes are strung together like beads on a string. While DNA that is tightly bound in nucleosomes or compacted into higher order heterochromatin is inaccessible and results in gene silencing, open euchromatin is accessible to binding and allows for active gene transcription (Jordan Rowley & Corces, 2018).

Chromatin is actively and dynamically remodeled to alter gene expression and cellular programming, for example, when immune cells are stimulated by antigens or other cells (Ramachandran et al., 2017). When this happens, large genomic regions can be silenced or activated, and nucleosomes are unraveled to access specific genes and DNA sequences, resulting in enhanced cytotoxicity of the immune cells. Molecular tools to study the 3D chromatin architecture reveal epigenetic programs and mechanisms involved in broad and specific cytotoxicity processes. Since chromatin architecture changes actively, cells need to be fixed to stabilize the protein-DNA interactions in the moment of fixation. The fixation protocol typically uses formaldehyde to covalently crosslink the DNA with its binding proteins in the nucleus.

DNA-protein interaction technology is vital in understanding the molecular mechanisms involved in gene expression, replication, and repair. Transcription factors (TFs), as a class of DNA-binding proteins, directly control the transcription of key regulators in major signaling pathways across the system.¹ Dysregulation in TFs or their binding sites plays a significant role in human diseases, particularly cancer pathogenesis.² Therefore, understanding the complex interactions between DNA and proteins is paramount in unraveling the intricacies of gene regulation, disease mechanisms and ultimately, drug discovery and personalized medicine.

The current methods of studying DNA-protein interaction center around Chromatin Immunoprecipitation Sequencing (ChIP-Seq). ChIP-seq is a powerful high-throughput technique that combines ChIP with next-generation sequencing to identify DNA-protein interactions across the entire genome. It offers a comprehensive view of the binding sites of a specific transcription factor.³ However, there are several potential sources of error within the ChIP-seq workflow. The technology requires a substantial amount of starting material, and the DNA fragments captured after sonication are heterogenous, posing challenges for analyzing DNA-protein interactions within a specific cell.⁴ Investigation at the single-cell level is essential, especially in immune responses, as sensitivity to therapy is influenced by cellular heterogeneity.⁵ Moreover, ChIP-seq lacks the capability to investigate DNA-transcription factor interactions during the functional processes of the cell.

DNA and protein interactions are essential for cellular processes and have major implications in cancer biology. DNA binding proteins, particularly transcription factors, are crucial in regulating gene expression and downstream protein functions. Studying the intricate DNA-protein interactions helps unravel specific functions of transcription factors that are involved in cancer pathogenesis, identify regulatory pathways, and ultimately uncover therapeutic targets. Currently, the lack of high-throughput screening techniques makes research in cancer associated-transcription factors challenging.

### SUMMARY

The present technology provides single-cell methods which can be used to investigate DNA-protein interactions and evaluate the cytotoxicity of immune cells towards target cells, such as tumor cells. The methods combine droplet microfluidics and imaging-based technology to facilitate the isolation and analysis of protein-bound DNA from individual apoptotic tumor cells, and can also be used to analyze similar interactions in individual immune effector cells.

In this technology, an effector cell is paired with a target cell at the single-cell level together with magnetic beads that are conjugated to antibodies useful to isolate the protein-bound DNA from the apoptotic target cell in droplets. Subsequently, the beads are released from droplets, and the genetic material captured and subjected to sequencing, such as by using ChIP-seq. This approach facilitates the precise analysis of DNA-protein interactions in apoptotic tumor cells, providing valuable insights into the molecular mechanisms that drive apoptosis in target cells. Additionally, this approach bypasses the need for a large amount of starting material and cell lysis via sonication, offering a time-efficient and cost-effective method for DNA-protein interaction analysis.

The present technology includes a novel protocol for applying ChIP-seq to genome-wide association studies (GWAS), utilizing microfluidic droplets containing single cell pairs and capture beads to pull out transcription factors and their bound DNA segments. This method, termed Iso-CUT&MAP (Isolated cell-based Cut Under Target and Mapping Apoptotic Protein-binding sequences), is unique in its ability to identify transcription factor activity of a cancer cell killed by an immune cell or chemotherapy. This provides a detailed understanding of transcription factor activity in response to treatment, something critical for understanding the response to treatment in the body. A validation study focused on Rel-A, a subunit of NF-κB. This provided an optimal target for validation of the Iso-CUT&MAP approach due to the expansive and variable range of effects NF-κB can have in cancer cell signaling, and its potential as a target for combination therapies. The K562 leukemia cell line and the NK92 cell line were chosen as a model effector-target pair, as K562 cellular response to NF-κB has previously been observed, and its susceptibility to NK92 cell mediated cytotoxicity is well documented.

One aspect of the present technology is a method for epigenetic analysis of one or more cytotoxic immune cells. The method includes the steps of: (a) providing a first aqueous suspension comprising cytotoxic immune cells, a second aqueous suspension comprising target cells, and a microfluidic device configured for co-encapsulating and imaging individual said cytotoxic immune cells with individual said target cells in aqueous microdroplets in an oil stream; (b) forming a plurality of said aqueous microdroplets, distributing and imaging the microdroplets in a plurality of docking stations in said microfluidic device, and incubating and imaging the microdroplets for a period of time; (c) selecting a portion of the microdroplets in which killing of a said target cell has occurred according to selected criteria; (d) collecting and merging the selected microdroplets; (e) fixing the contents of the merged microdroplets with a chemical fixing agent, whereby cytotoxic immune cells are fixed; (f) lysing the fixed cells and isolating nuclei of the fixed cytotoxic immune cells; (g) immobilizing the isolated nuclei on a solid support; (h) incubating the immobilized nuclei with an antibody that specifically binds to a selected nucleic acid-binding protein; (i) incubating the immobilized nuclei with a nuclease, whereby nucleic acid not bound to the selected nucleic acid-binding protein is degraded, leaving nucleic acid fragments that were bound to the selected nucleic acid-binding protein; (j) isolating said nucleic acid fragments; and (k) sequencing one or more of the isolated nucleic acid fragments, whereby one or more nucleic acids interacting with the selected nucleic acid-binding protein in said one or more cytotoxic immune cells are identified.

Another aspect of the technology is a method of epigenetic analysis of one or more target cells following interaction between said one or more target cells and one or more cytotoxic immune cells. The method includes the steps of (a) providing a first aqueous suspension comprising cytotoxic immune cells, a second aqueous suspension comprising target cells and microbeads coated with a capture agent for a selected target cell nucleic acid-binding protein, and a microfluidic device configured for co-encapsulating and imaging individual said cytotoxic immune cells with individual said target cells in aqueous microdroplets in an oil stream; (b) forming a plurality of said aqueous microdroplets, distributing and imaging the microdroplets in a plurality of docking stations in said microfluidic device, and incubating and imaging the microdroplets for a period of time, whereby in a portion of the microdroplets a said target cell has been killed by a said cytotoxic immune cell, and whereby the microbeads bind to the nucleic acid-binding protein in the killed target cells; (c) selecting a portion of the microdroplets in which killing of a said target cell has occurred according to selected criteria; (d) collecting and merging the selected microdroplets; (e) isolating said microbeads; (f) releasing from the isolated microbeads nucleic acid fragments that were bound to said nucleic acid-binding protein; (g) isolating said nucleic acid fragments; and (h) sequencing one or more of the isolated nucleic acid fragments, whereby one or more nucleic acids interacting with the nucleic acid-binding protein in said one or more target cells are identified.

Yet another aspect of the present technology is a kit for epigenetic analysis of a cytotoxic immune cell. The kit includes (i) a microfluidic device configured for co-encapsulating and imaging individual said cytotoxic immune cells with individual said target cells in aqueous microdroplets in an oil stream; and (ii) an antibody that specifically binds a selected nucleic acid binding protein of the cytotoxic immune cell.

Still another aspect of the present technology is a kit for epigenetic analysis of a target cell. The kit includes (i) a microfluidic device configured for co-encapsulating and imaging individual said cytotoxic immune cells with individual said target cells in aqueous microdroplets in an oil stream; and (ii) a plurality of microbeads comprising a capture agent, such as an antibody or aptamer, that specifically binds a selected nucleic acid binding protein of the target cell.

The technology can be further summarized in the following list of features.

1. A method for epigenetic analysis of one or more cytotoxic immune cells, the method comprising the steps of:
(a) providing a first aqueous suspension comprising cytotoxic immune cells, a second aqueous suspension comprising target cells, and a microfluidic device configured for co-encapsulating and imaging individual said cytotoxic immune cells with individual said target cells in aqueous microdroplets in an oil stream;
(b) forming a plurality of said aqueous microdroplets, distributing and imaging the microdroplets in a plurality of docking stations in said microfluidic device, and incubating and imaging the microdroplets for a period of time;
(c) selecting a portion of the microdroplets in which killing of a said target cell has occurred according to selected criteria;
(d) collecting and merging the selected microdroplets;
(e) fixing the contents of the merged microdroplets with a chemical fixing agent, whereby cytotoxic immune cells are fixed;
(f) lysing the fixed cells and isolating nuclei of the fixed cytotoxic immune cells;
(g) immobilizing the isolated nuclei on a solid support;
(h) incubating the immobilized nuclei with an antibody that specifically binds to a selected nucleic acid-binding protein;
(i) incubating the immobilized nuclei with a nuclease, whereby nucleic acid not bound to the selected nucleic acid-binding protein is degraded, leaving nucleic acid fragments that were bound to the selected nucleic acid-binding protein;
(j) isolating said nucleic acid fragments; and
(k) sequencing one or more of the isolated nucleic acid fragments, whereby one or more nucleic acids interacting with the selected nucleic acid-binding protein in said one or more cytotoxic immune cells are identified.
   2. The method of feature 1, wherein the one or more cytotoxic immune cells comprise NK cells.
   3. The method of feature 1 or feature 2, wherein the target cells comprise tumor cells or cells infected with a microbial pathogen.
   4. The method of any of the preceding features, wherein the incubation period of time is sufficient to allow killing of at least a portion of the target cells by the co-encapsulated cytotoxic immune cells.
   5. The method of any of the preceding features, wherein the selected criteria of step (c) comprise elapsed time to killing of a target cell.
   6. The method of any of the previous features, wherein the selected nucleic acid-binding protein is a transcription factor.
   7. The method of any of the preceding features, wherein the nuclease is micrococcal nuclease.
   8. The method of any of the preceding features, wherein step (j) further comprises amplifying the nucleic acid fragments, such as by polymerase chain reaction or rolling circle amplification.
   9. The method of feature 8, wherein the epigenetic analysis is performed on a single cytotoxic immune cell.
   10. The method of any of the preceding features, wherein step (j) further comprises preparing a library of said nucleic acid fragments.
   11. The method of any of the preceding features, wherein said nucleic acid comprises DNA, RNA, or both.
   12. A method of epigenetic analysis of one or more target cells following interaction between said one or more target cells and one or more cytotoxic immune cells, the method comprising:
(a) providing a first aqueous suspension comprising cytotoxic immune cells, a second aqueous suspension comprising target cells and microbeads coated with a capture agent for a selected target cell nucleic acid-binding protein, and a microfluidic device configured for co-encapsulating and imaging individual said cytotoxic immune cells with individual said target cells in aqueous microdroplets in an oil stream;
(b) forming a plurality of said aqueous microdroplets, distributing and imaging the microdroplets in a plurality of docking stations in said microfluidic device, and incubating and imaging the microdroplets for a period of time, whereby in a portion of the microdroplets a said target cell has been killed by a said cytotoxic immune cell, and whereby the microbeads bind to the nucleic acid-binding protein in the killed target cells;
(c) selecting a portion of the microdroplets in which killing of a said target cell has occurred according to selected criteria;
(d) collecting and merging the selected microdroplets;
(e) isolating said microbeads;
(f) releasing from the isolated microbeads nucleic acid fragments that were bound to said nucleic acid-binding protein;
(g) isolating said nucleic acid fragments; and
(h) sequencing one or more of the isolated nucleic acid fragments, whereby one or more nucleic acids interacting with the nucleic acid-binding protein in said one or more target cells are identified.
   13. The method of feature 12, wherein the capture agent is an antibody or an aptamer.
   14. The method of feature 12 or feature 13, wherein the one or more cytotoxic immune cells comprise NK cells.
   15. The method of any of features 12-14, wherein the target cells comprise tumor cells or cells infected with a microbial pathogen.
   16. The method of any of features 12-15, wherein the incubation period of time is sufficient to allow killing of at least a portion of the target cells by the co-encapsulated cytotoxic immune cells.
   17. The method of any of features 12-16, wherein the selected criteria of step (c) comprise elapsed time to killing of a target cell.
   18. The method of any of features 12-17, wherein the selected nucleic acid-binding protein is a transcription factor.
   19. The method of any of features 12-18, wherein step (g) further comprises amplifying the nucleic acid fragments, such as by polymerase chain reaction or rolling circle amplification.
   20. The method of feature 19, wherein the epigenetic analysis is performed on a single target cell.
   21. The method of any of features 12-20, wherein step (g) further comprises preparing a library of said nucleic acid fragments.
   22. The method of any of features 12-21, wherein said nucleic acid comprises DNA, RNA, or both.
   23. The method of any of features 12-27, wherein the microbeads are magnetic, and a magnet is used to isolate the microbeads in step (e).
   24. The method of any of the preceding features, wherein the cytotoxic immune cells are NK cells and the target cells are tumor cells or cells infected with a microbial pathogen, and both types of cells are obtained from the same subject.
   25. The method of any of the preceding features, wherein two or more different antibodies that bind to two or more different nucleic acid binding proteins, or two or more different types of microbeads, each type of microbeads having a different said capture agent, are used, and the method is carried out in a multiplex format.
   26. A kit for epigenetic analysis of a cytotoxic immune cell, the kit comprising:
(i) a microfluidic device configured for co-encapsulating and imaging individual said cytotoxic immune cells with individual said target cells in aqueous microdroplets in an oil stream; and
(ii) an antibody that specifically binds a selected nucleic acid binding protein of the cytotoxic immune cell.
   27. The kit of feature 31, further comprising one or more enzymes and/or one or more reagents.
   28. A kit for epigenetic analysis of a target cell, the kit comprising:
(i) a microfluidic device configured for co-encapsulating and imaging individual said cytotoxic immune cells with individual said target cells in aqueous microdroplets in an oil stream; and
(ii) a plurality of microbeads comprising a capture agent, such as an antibody or aptamer, that specifically binds a selected nucleic acid binding protein of the target cell.
   29. The kit of feature 28, further comprising one or more enzymes and/or one or more reagents.

### BRIEF DESCRIPTION OF THE DRAWINGS

FIG. 1 shows a schematic representation of an embodiment of interactions between an immune effector cell (NK cell) and a target cell (cancer cell). The present technology provides methods for identifying and analyzing interactions between nucleic acid binding proteins, such as NFkB, with DNA or RNA in the cytosol or in the nucleus of both immune effector cells and target cells.

FIG. 2 shows a schematic representation of a Cut & Run analysis, which requires thousands of cells. The present technology modifies this approach by utilizing aqueous microdroplets in a microfluidic device such that individual cells or pairs of interacting immune cells and target cells can be analyzed to characterize interactions between nucleic acids and specific nucleic acid binding proteins.

FIG. 3 presents a flow diagram of an embodiment of a method for using a Cut & Run process with pairs of single immune cells interacting with single target cells.

FIG. 4A illustrates a strategy for studying interaction between single effector cells and target cells using a microfluidic device. The device (center of the figure) includes a cell mixing and droplet formation section, a docking section for incubation and imaging, and valves allowing harvesting of selected droplets, such as those with strong target cell killing activity meeting pre-selected criteria. FIG. 4B depicts the use of the microfluidic device for incubating cell pairs in docked droplets, monitoring cell interactions, and selective release of droplets. See Agnihotri, et al., 2022, Lab Chip 22, 3258.

FIG. 5 is a flow diagram for an embodiment of a method for using an Iso Cut & Map process with pairs of single immune cells interacting with single target cells for analysis of genetic interactions with nucleic acid binding proteins in apoptotic target cells.

FIG. 6A is a schematic diagram of a Cut & Map protocol for analysis of individual apoptotic target cells. Effector cell 110 is co-encapsulated with target cancer cell 120 and antibody-conjugated magnetic capture bead 130 in microdroplet 140. These cells are incubated for enough time for effector cells to kill target cells, after which proteins of interest 150 that are conjugated to DNA or RNA are captured by the antibody-conjugated beads. Droplet emulsions collected from the microfluidic device are then merged, and beads are collected by magnetic separation 160. FIG. 6B represents a multiplexed version of the same protocol, for use in analysis of both RNA and DNA simultaneously, in the depicted embodiment.

FIG. 7A shows a drawing (left) depicting a single-cell droplet microfluidic device and microscope images (right) showing cell and bead droplet co-encapsulation. FIG. 7B shows a line plot depicting viability of K562 cells in the presence (lower curve) or absence (upper curve) of NK92 cells. FIG. 7C is a scatter plot showing the precise times of death of K562 cells co-encapsulated with NK92 cells.

FIGS. 8A-8B show results of a genome wide association study (GWAS) of Rel-A/p65. FIG. 8A shows mapping of ChIP-seq peaks across the entire genome, by chromosome. FIG. 8B shows mapping of ChIP-seq summits across the entire genome, by chromosome.

FIG. 9A is a distribution plot of fragment size and density based on a kernel density method. High density (center section of peak) is around the mean, and low density is at the ends (left and right shoulders of the peak). SD=standard deviation. FIG. 9B is a distribution plot of peak width and the number of the corresponding peaks. FIG. 9C is a distribution plot of the distance (bp) from peak to the transcription start site (TSS). FIG. 9D shows the distribution of the ratio of mapped reads/total reads in 2 kb upstream (left hand portion of curve) and 2 kb downstream (right hand section) of the gene body (center section).

FIGS. 10A-10B show results of analysis of genes influenced by Rel-A binding-based GO and KEGG databases. FIG. 10A is a bar graph summarizing Gene Ontology (GO) analysis. The x-axis represents the total number of genes related to each GO term. FIG. 10B is a bar graph summarizing Kyoto Encyclopedia of Genes and Genomes (KEGG) terms of interest. The x-axis represents-log 10 of corrected p-values of statistical significance for each KEGG term.

FIG. 11 is a heatmap representing the log 2 adjusted transcriptomic expression of certain genes of interest. K562 and NK92 were encapsulated in droplets and incubated prior to RNA extraction and sequencing.

FIG. 12 shows results of mRNA extraction and sequencing mRNA. The data show integrity and classification of reads and their respective genome regions.

### DETAILED DESCRIPTION

The present technology makes use of microfluidics approaches to co-encapsulate immune cells with their target cells and to characterize immune cell cytotoxicity levels and target cell responses at the level of epigenetics. The aftermath of each single cell co-encapsulation experiment includes live cytotoxic immune cells and the cytoplasmic and nucleic debris of dead target cells. The present technology enables study of the interaction of DNA and/or RNA with regulatory binding proteins, such as transcription factors and other gene regulatory or packaging proteins. Analysis of single immune cells based on the isolation of their nuclei from co-encapsulated immune cell-target cell samples with the aid of a microfluidic system makes it possible to characterize cell-cell interactions and collect cells of interest. With similar methods, analysis of single target cells can be carried out to probe mechanisms of target cell sensitivity and apoptosis.

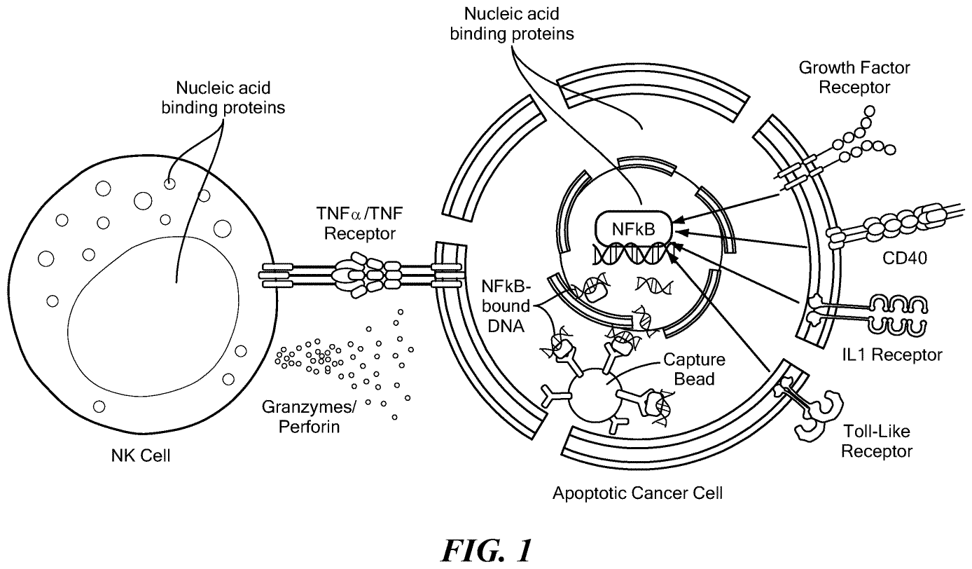

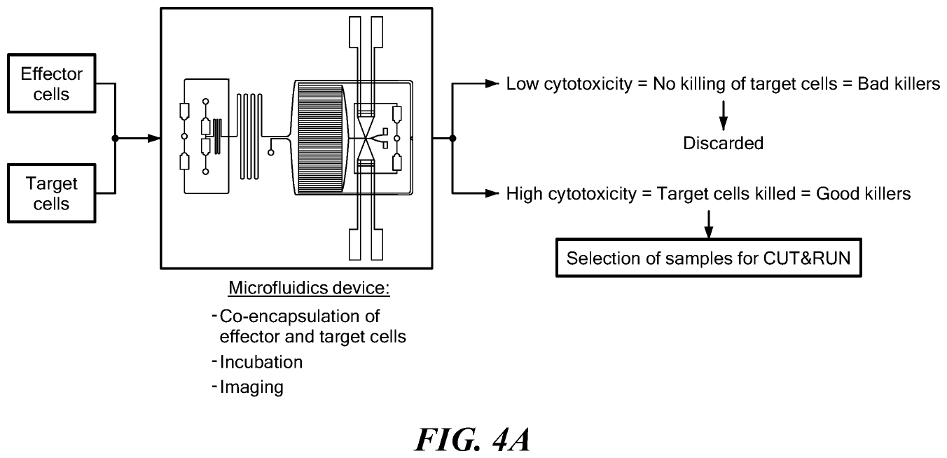

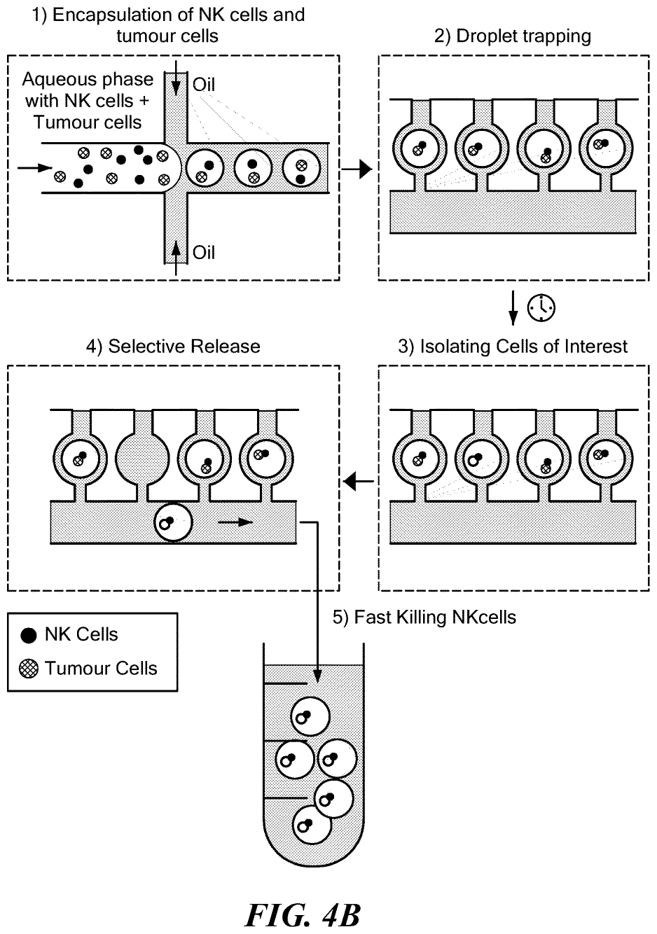

To study the cytotoxicity properties of effector cells, both effector cells (i.e., immune cells such as T cells and natural killer (NK) cells) and target cells (such as tumor cells or pathogen-infected cells) are incubated together for a period of time sufficient to allow killing of target cells; this can be done in bulk or in single-cell droplets using a microfluidic system. FIG. 1 depicts the interaction between a pair of effector and target cells. In the single-cell microfluidic-based assay, the sample droplets are the ones selected after lytic effects have been proven by imaging (Agnihotri et al., 2022). FIG. 4A shows an overall strategy that can be employed to select fast killing NK cells, for example, from a group of individual cell pairs docked and observed in a microfluidic device. FIG. 4B shows how a suitable microfluidic device can form aqueous microdroplets containing cell pairs in an oil stream, store them in observable docking stations, and release selected droplets individually after observation using pneumatically controlled microvalves. For further details of such a microfluidic device and droplet harvesting, see Agnihotri et al., 2022, which is hereby incorporated by reference.

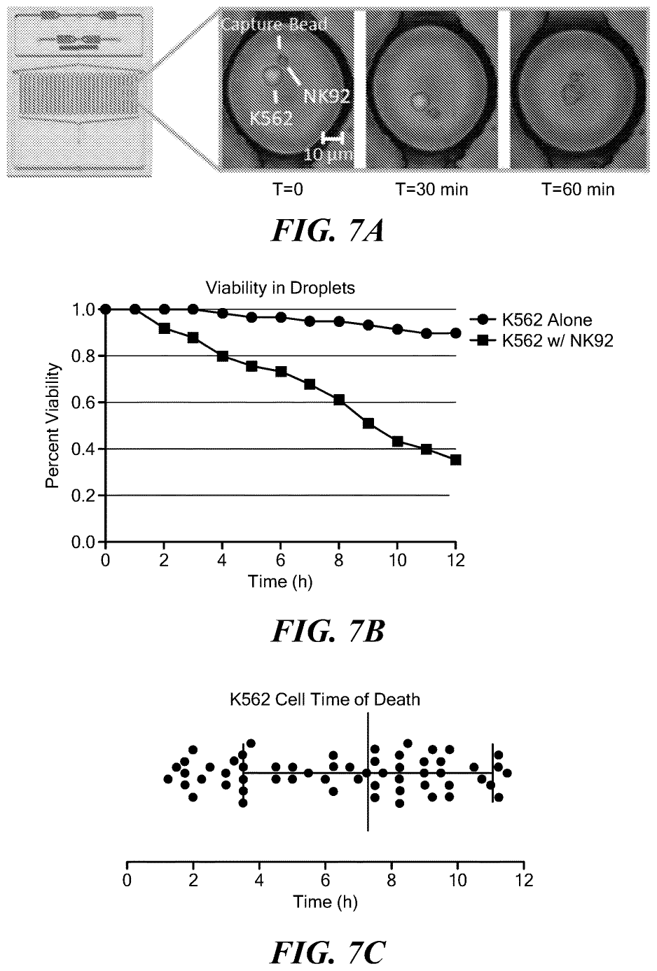

Once the effector cells interact with the target cells, their cytolytic activity gets activated and results in the destruction of the target cells and their nuclear contents. The remaining droplet sample includes the live effector cells and the cytoplasmic and nucleic debris of the dead target cells (see FIG. 7A). The isolation of whole nuclei after fixation ensures that only the living effector cells nuclei are being separated and thus, the cytoplasmic and nucleic debris of dead target cells do not interfere with the subsequent epigenomic analysis.

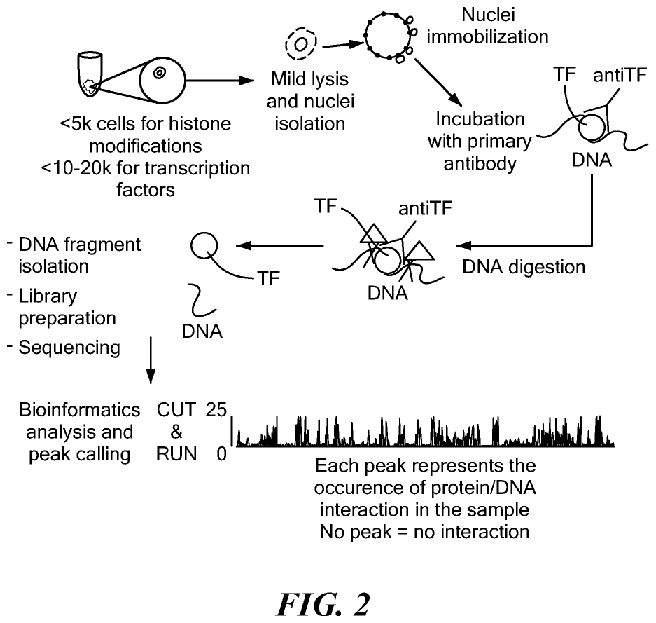

In an embodiment, once the fixed nuclei are isolated, the method follows the Cleavage Under Targets and Release Using Nuclease (CUT&RUN) protocol (Janssens & Henikoff, 2019; Skene & Henikoff, 2017). This protocol is a chromatin profiling strategy in which antibody-targeted controlled cleavage releases specific protein-DNA complexes into the supernatant for sequencing (FIG. 2) (Yu et al., 2022). Analysis of the sequences provides information about the epigenetic interactions and gene expression levels of immune cells that can be correlated with their cytotoxicity (Meers et al., 2019).

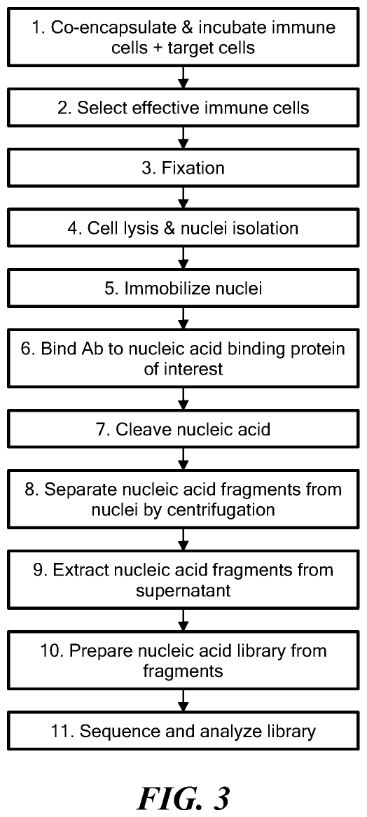

The adaptation of CUT&RUN used in the present technology involves three main protocols. The first protocol is the co-encapsulation and incubation of immune and target cells in a single-cell microfluidic device. The second protocol is the fixation and isolation of intact cytotoxic immune cell nuclei followed by performing the CUT&RUN process. The third protocol involves the sequencing and analysis of the resulting DNA fragments. An example of the full procedure is shown in FIG. 3.

In an embodiment, a CUT&RUN adaptation of the present technology involves the following 11 steps, Steps 1 and 2 are performed in the a microfluidic device, while steps 3 to 9 are steps of the basic CUT&RUN process; and steps 10 and 11 are the sequencing and analysis of the genomic information. Step (1) is the co-encapsulation and incubation of immune and target cells. Step (2) is selection of the cytotoxic cells based on imaging data from step 1. Step (3) is fixation of the samples with formaldehyde. Step (4) is hypotonic lysis of the cells and isolation of nuclei by centrifugation. Step (5) is the immobilization of nuclei on lectin-coated magnetic beads. Step (6) is the incubation with an antibody targeted at the DNA-binding protein of interest. Step (7) is the incubation with a nuclease to cleave the DNA around the DNA-binding protein of interest. In step (8), the cleaved DNA fragments are liberated into the supernatant after the nuclei are pelleted by centrifugation; In step (9), the DNA fragments are then extracted from the supernatant. Step (10) involves using a kit to prepare a sequencing library from the extracted DNA fragments. Step (11) is the sequencing and analysis of the genomic material of the library.

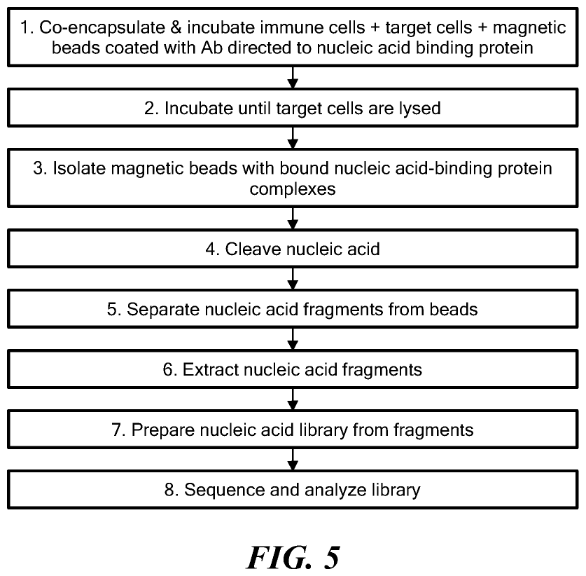

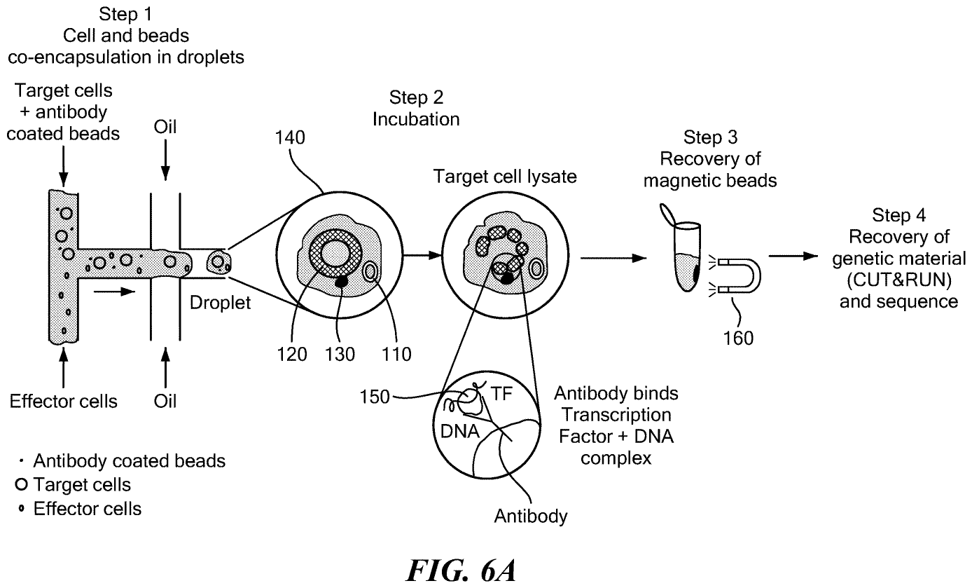

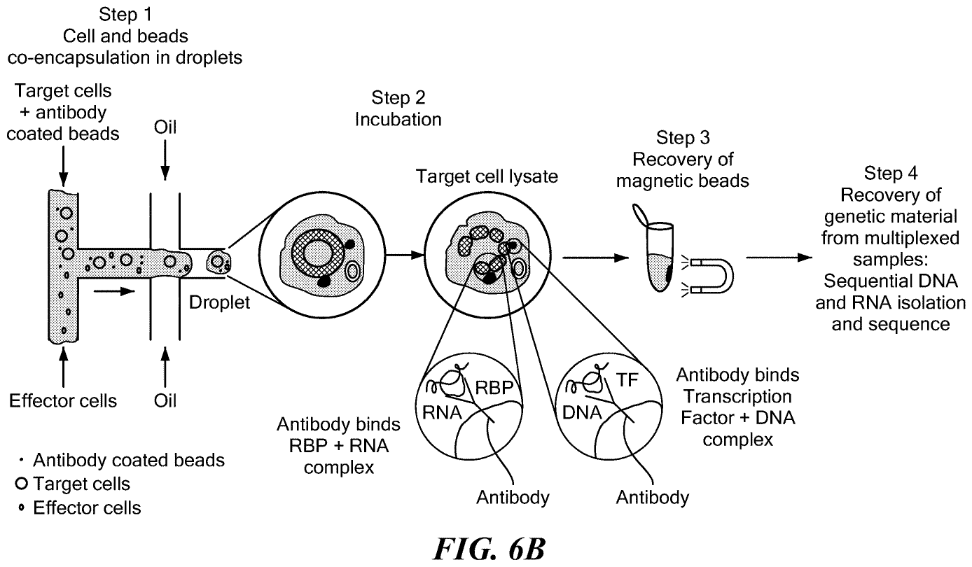

In another embodiment, genetic material is collected from dead target cells that have undergone apoptosis, and the genetic material is analyzed. See FIGS. 6A-6B. This process can be referred to as Iso-CUT&MAP (Isolated cell-based Cut Under Target and Mapping Apoptotic Protein-binding sequences). The cytotoxicity of effector cells induces apoptosis in the target cells attacked by the effector cells, and the apoptosis results in the fragmentation of the genomic material at the end of the process (He et al., 2009; Hu et al., 2021; Majtnerová & Roušar, 2018). The fragments of genetic material can be collected by targeting the proteins interacting with them with one or more antibodies specific for the nucleic acid-associated proteins. This method avoids isolation of the genetic material from the immune cells because it targets the dead cells debris in the aqueous microdroplet. The antibodies can be attached to a solid support, such as microbeads, which can be magnetic microbeads, thereby allowing the fragments of apoptotic target cells to be simply harvested without disrupting any non-apoptotic cells, such as the effector cells, present in the incubation mixture. This method can include the following four steps (for a more detailed breakdown see FIG. 5).

In step 1, cells and beads are co-encapsulated in aqueous microdroplets containing single-cells using a microfluidic device. Preferably, each droplet contains one effector cell, one target cell, and one or more antibody-coated microbeads. The antibodies are directed to one or more transcription factors expected to be interacting with fragmented genetic material of the target cells.

In step 2, the co-encapsulated cells are incubated, leading to lysis of the target cells. At the end of this step, the antibody-coated beads capture transcription factor-DNA complexes from the dead target cells.

In step 3, the magnetic beads are recovered from the microdroplets, for example, by using a magnetic rack.

In step 4, the genetic material is isolated and analyzed using a CUT&RUN method and sequencing.

In another embodiment, RNA from one or more apoptotic target cells is isolated and sequenced. Effector cell cytotoxicity induces apoptosis in target cells, and thus the cytosolic content including RNA is released and degraded (Hu et al., 2021). In single-cell droplets generated in a microfluidic device, the environment can be controlled by adding RNAse inhibitors and RNA degradation can be prevented. In this method, RNA bound to RNA-binding proteins (RBPs) from dead target cells is isolated with an antibody specific for an RNP. RBPs are a family of proteins that specifically bind to RNAs and are involved in post-transcriptional regulation of gene expression and important cellular processes. In cancer cells, RBPs play an important role in regulating the expression of tumor suppressors and oncoproteins involved in various cell-signaling pathways (Mehta et al., 2022). The present method avoids isolation of the genetic material from immune cells because it targets the dead cell debris in the droplet.

A four step method can be used to isolate and sequence the RNA from dead target cells using antibody coated beads to bind RBPs (FIG. 6A). In a variation of this method, a multiplexed assay is used to sequentially isolate both RNA and DNA from the same cell using both types of beads simultaneously (FIG. 6B). Both types of beads are magnetic to allow for isolation of the beads.

Step 1. Cells and beads are co-encapsulated in droplets containing single-cells (i.e., a small number of individual cells) using a microfluidic device. Each droplet selected for analysis preferably contains one effector cell, one target cell, and one or more beads. RNAse inhibitors are added to the culture medium.

Step 2. The droplets are Incubated and observed in the microfluidic device, until the target cell is lysed. Following lysis, the antibody coated beads capture the RBP-RNA complexes, and in the multiplexed assay a second type of antibody-coated beads also capture the transcription factor-DNA complexes, from the dead target cell Step 3. The magnetic beads are recovered from the droplets using a magnetic rack.

Step 4. The RNA, and in the multiplexed assay also the DNA, is Isolated and sequenced. Preferably, in the multiplexed format, RNA and DNA are isolated sequentially and then sequenced separately.

To validate the Iso-CUT&MAP method, we utilized RelA, an optimal target protein in tumor cell apoptosis, as it is a component of the most frequently binding form of NFkB in its canonical signaling pathway.⁶ See Examples 1-10 below. RelA is the primary subunit in regulating the induction or inhibition of target gene transcription. By collecting RelA-bound DNA from cancer cells that were killed by immune cells, the genome-wide activity of NFkB in response to immune cytotoxicity can be seen. Iso-CUT&MAP was used to observe RelA genome-wide association in K562 cancer cells in response to cytotoxicity from NK92 effector cells. The genome-wide mapping matched the expected profile of a transcription factor, indicating an accurate representation of NFkB. Additionally, hits were observed on several genes known to interact with NFkB; genes which are both correlated to susceptibility and resistance to killing. While the results matched an overall resistant phenotype as would be expected for K562 cells, interestingly, RelA was found to interact with multiple genes that potentially promote apoptosis. Overall, the Iso-CUT&MAP approach can be employed to elucidate the complex transcription factor-regulated pathways in cancer cells and their response to treatment. Using this method, researchers can gain insights into the dynamic interactions and mechanisms underlying cancer cell behavior, paving the way for targeted interventions in cancer research.

The ChIP-seq results confirmed a GWAS profile in line with known properties of NF-κB. This was further confirmed by looking at fragment size, peak width, and Rel-A binding site, which all matched expectations of a transcription factor such as NF-κB. To take a deeper look at the overall impact of NF-κB in K562 cells, the specific genes found in our ChIP-seq results were examined. GO and KEGG terms listed many pathways related to cellular activity, including response to stress as well as apoptosis. Looking at the specific genes related to these terms, an overall trend towards resistance to apoptosis in NF-κB was found, as has been previously shown in K562 cells. Some pathways related to resistance were also found to be upregulated, indicating a mixed response overall to NF-κB. Transcriptomic sequencing of cells alone and co-encapsulated in droplets was performed to compare expression levels to ChIP-seq results. Expression of mRNA matched expectations for the genes of interest found in association with Rel-A, supporting the accurate mapping of Rel-A activity in these cells.

The results obtained by the inventors reinforce the accuracy of Iso-CUT&MAP for identifying transcription factor binding in response to immune cell interactions. The approach is more efficient and reliable than some previously documented methods of GWAS extraction, which can require tedious DNA isolation processes and may provide less accurate representations of transcription factor activity by pulling protein-bound DNA out of bulk culture at random. In Iso-CUT&MAP, using cancer cells killed by treatment and beads co-encapsulated in droplets reduces steps, and ensures a full, unbiased sampling of protein-bound DNA from every cell. Additionally, observing transcription factor binding specifically in cancer cells that have been killed by immune cells or therapeutics provides a unique observation of how the transcription factor influences cellular response to treatment. This method provides a useful tool for characterizing the response of NF-κB and other transcription factors of interest. Understanding the exact influence of treatments on transcription changes, provides unique insights into the complex biology of cancer cells, and can help further refine therapeutic strategies.

The novel and unusual features of the present technology include the following.
(1) Analysis of genomic information derived from live immune cells from a sample containing both live immune cells and dead target cells.
(2) The technology can be used with more than one antibody per sample, so as to select more than one protein of interest that binds specific DNA and/or RNA sequences.
(3) The technology allows identification of subpopulations of immune cells based on their epigenome to better understand their cytotoxic capabilities.

The advantages of the present technology over previous technology include the following.
(1) Isolation of cell-specific genomic material from samples containing both live immune cells and dead target cells.
(2) Identification of new genomic interactions associated with cytotoxicity of immune cells.
(3) The technology allows identification of new genomic targets that can be used in immune cell-based therapies.

Uses of the present technology include the following.
(1) Profiling of patient immune and cancer cells to study their specific epigenomic modifications. The technology can be used for precision medicine, in which the process of apoptosis and the influence of immune cell or chemotherapy-induced tumor cell killing are analyzed for the individual patient, or to develop new treatments or new drugs.
(2) Performing multi-omic analyses, such as ATACseq, transcriptomics, qPCR.
(3) Identification of new subpopulations of cytotoxic immune cells.
(4) Identification of new protein-DNA interactions.
(5) Identification of DNA binding proteins and/or domains.

### EXAMPLES

### Example 1. Bead and Capture Antibody Preparation

Carboxylated magnetic microspheres (PolyLink Protein Coupling Kit from Bang Laboratories Inc., IN) were covalently coupled with an antibody against NF-κB (ChlpAB+NF-κB p65 (RelA)-ChIP validated antibody and primer set from Sigma-Aldrich, MO; forward primer: GACGACCCCAATTCAAATCG (SEQ ID NO:1), reverse primer: TCAGGCTCGGGGAATTTCC (SEQ ID NO:2). The beads were treated and washed with 0.1M MES buffer pH 5.2 (Coupling Buffer) and then treated with 2% carbodiimide 0.1M ME PH 5.7 and 1.2 g of NF-κB p65 antibody. After incubation, the coated beads were resuspended and stored in 10 mM Tris pH 8; 0.05% bovine serum albumin (Wash/Storage buffer).

### Example 2. Cell Isolation and Culture

K562 cells (lymphoblast cells from chronic myelogenous leukemia) were purchased from American Type Culture Collection (ATCC, Manassas, VA) and maintained in RPMI-1640 medium supplemented with 10% fetal bovine serum and 1% antibiotic-antimycotic solution (Corning Cellgro, Manassas, VA). NK-92 cells were obtained from ATCC and maintained in X-Vivo 10 medium (Lonza, NJ) supplemented with 5% human serum and 500 IU/mL IL-2 (ProSpec Bio, East Brunswick, NJ). All cells were grown at 37° C. under 5% CO₂ in a humidified atmosphere. K562 cells were passaged every two days and harvested at a density of 1×10⁶ viable cells/mL, while NK92 cells were passaged every four days and harvested at a density of 1×10⁶ viable cells/mL. For the co-encapsulation protocol, K562 cells were loaded in a syringe at an initial concentration of 1 million/mL and NK92 cells were loaded in a separate syringe at a concentration of 3 million/mL.

### Example 3. Microfluidic Device Fabrication

Microfluidic devices were fabricated by standard soft-lithography protocols. 7,8 The microfluidic design was etched on silicon wafers with a negative photo resist SU-8 2100 (MicroChem, Newton, MA) to obtain features of 150 μm height. Poly(dimethylsiloxane) (PDMS) (Sylgard 184, Dow Corning, Midland, MI) devices were prepared by mixing the pre-polymer with silicone elastomer curing agent at 10:1 ratio (w/w) and poured over the wafer. The mixture was degassed and cured for 12 hours at 65° C. Individual PDMS devices were peeled from the wafer, bonded to microscope slides by heating at 90° C. for 10 minutes. Each inlet of the device was connected to an individual syringe containing aqueous (i.e., cell suspension in culture medium) or oil-based fluids through Tygon Micro Bore PVC tubing (0.010″ ID, 0.030″ OD, 0.010″ wall, Small Parts Inc., FL, USA). The device was treated with Aquapel glass treatment (Aquapel, Pittsburgh, USA), then dried at room temperature immediately before experiments. The syringes were operated by individually programmable syringe pumps (Harvard Apparatus, USA).

### Example 4. Droplet Generation and Incubation

To obtain optimal droplet sizes the oil to aqueous flow rates were generally maintained at a ratio of 4:1 in the device. The oil phase consisted of Fluorinert® FC-40 (Sigma, St. Louis, MO) supplemented with 2% w/w surfactant (008-FluoroSurfactant, Ran Biotechnologies, Beverly, MA). The water-in-oil droplets were generated upstream using a flow-focusing junction. The aqueous phase contained NK cells and tumor cells with beads mixed before reaching a flow-focusing junction at 200 μl per hour. FC-40 with 2% surfactant Span-80 was used as a continuous phase at 1000 μl per hour. The oil and aqueous phases were pumped into the microfluidic device using three syringe pumps. Under the flow-focusing geometry dimensions, droplets having a diameter in the range of 150 to 200 μm were generated. These generated droplets were collected in a sterile 15 ml tube containing mineral oil to prevent evaporation The collected droplets encapsulating NK cells and tumor cells with beads were incubated at 37° C., 5% CO₂ for 6 hrs. These parameters were optimized based on a standard single-cell cytotoxicity assay as described.⁹

### Example 5. DNA Isolation and Sequencing

After immune cells, target cells and (NF-κB) conjugated beads were co-encapsulated and incubated for 6 hours, the time needed to observe sufficient killing of K562 by NK92 cells, the antibody coated beads, capturing the transcription factor interacting with the DNA from the dead target cell (transcription factor+DNA complex), were pulled out from the droplets using a magnetic rack (0.5-0.7 mL tube Magnetic Rack, Amazon) for 10 minutes. The beads were washed with 0.1M MES buffer at pH 5.2 (Coupling Buffer), and a simple DNA isolation protocol was performed using a DNA Clean & Concentrator™-5 (Cat #D4014, Zymo Research Corporation).

### Example 6. Sequencing Using ChIP-Seq

Genomic DNA underwent pair-end ChIP-seq using Illumina NovaSeq 6000 at Novogene Co. (Durham, NC). The Skewer feature was used to filter raw data. The procedure for data trimming was the following: (1) discard the reads with low quality (proportion of low quality bases larger than 50%); (2) discard the reads with N ratio (unsure base) larger than 15%; (3) discard the reads with adaptor at the 5′-end; (4) discard the reads without adaptor and inserted fragment at the 3′-end; (5) trim the adapter sequence at the 3′-end; (6) discard the reads whose lengths are less than 18 nt after trimming. Base-calling was performed using Illumina CASAVA v1.8 software. Read alignment software was BWA. Genome assembly was performed using grch38.

### Example 7. Droplet Microfluidics and Iso-CUT&MAP Technology

After a lytic hit by immune cells, tumor cells become apoptotic and develop membrane holes.^{10,11} In addition, during apoptosis the nuclear envelope breaks down, allowing for the release of fragmented DNA into the cytoplasm which facilitates access to DNA within the affected cells.¹²⁻¹⁴ This occurrence can potentially provide opportunities for access and further analysis or manipulation of the genetic material of an apoptotic cell.

The inventors developed a droplet microfluidic integrated method called iso-CUT&MAP to isolate and analyze genetic material of single apoptotic target cells. The method utilized the combination of droplet microfluidics and imaging-based methods, where effector and target cells at the single cell level were co-encapsulated and protein-bound DNA segment was isolated for sequencing analysis (FIG. 6A).

RelA(p65) from the NF-κB transcription factor family was used to test the approach, as downregulated NF-κB has been linked to a variety of human diseases, particularly cancer biology.¹⁵ The NF-κB family consists of five closely related DNA binding proteins, RelA(p65), RelB, c-Rel, NF-κB1/p50 and NF-κB2/52, that have functions as various homodimers and heterodimers in the NF-κB signaling pathways.¹⁵ Among these subunits, RelA/p65 plays a critical role in the NF-κB complex, where it is responsible for DNA binding dynamics and the regulation of gene expression in several disease pathophysiology.

K562 target cells and NK-92 effector cells were co-encapsulated within the droplet microfluidics device, and isolated NF-κB bound-DNA segments were used for downstream sequencing analysis (FIG. 7A). K562 viability had a relatively linear decrease over the first 8 hours of co-culture, with about 40% killed by NK-92 by the end of 8 hours (FIGS. 7B-7C). K562 cells alone, meanwhile, maintained viability at about 90% for 8 hours. Based on these data, a 6-hour incubation time was chosen for Iso-CUT&MAP experiments to observe adequate K562 killing by NK92 cells with minimal spontaneous cell death.

### Example 8. Enrichment of Rela/p65 (NF-κB) Binding Regions

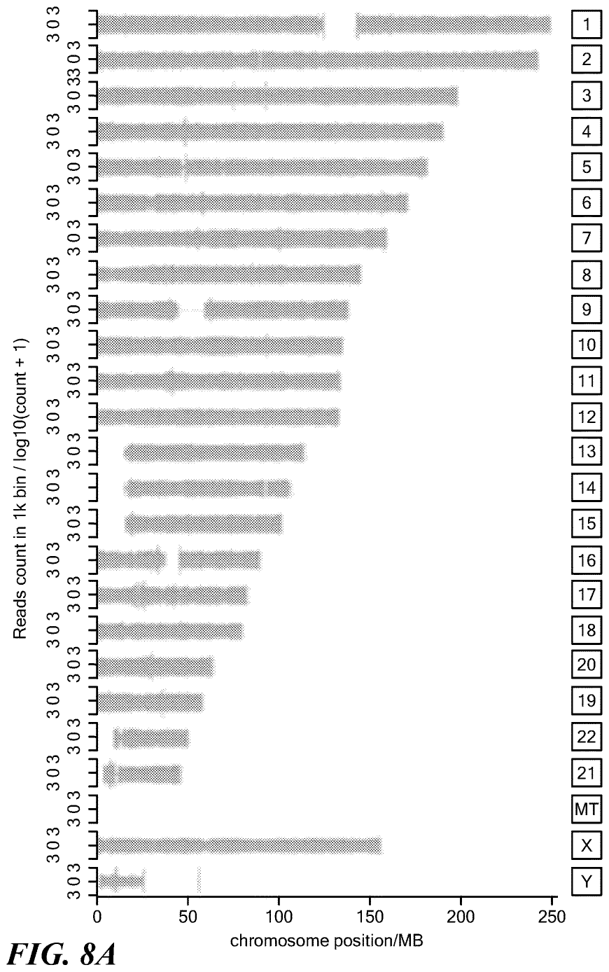

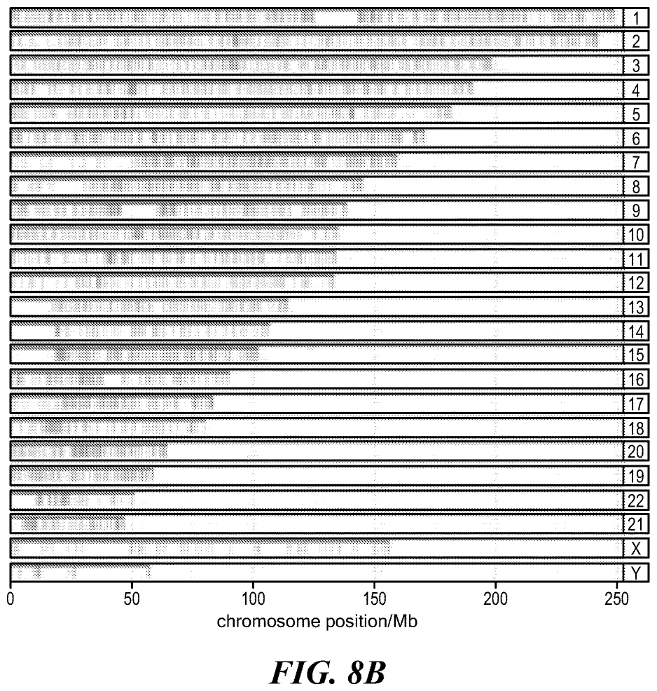

To identify the specific regions of NF-κB protein binding in the genome, ChIP-seq was performed to observe the distribution of Rela/p65 binding regions in all chromosomes. The identification and mapping of NF-κB binding sites using our Iso-CUT&MAP approach can aid in exploring the regulatory roles of NF-κB in cancer cell survival and proliferation. Peaks representing general regions of the genome where NF-κB is bound are observed across most chromosomes (FIG. 8A). The number of sequenced reads that aligned to these regions varied as indicated by the height of the peaks, suggesting that some areas of the genome are more regulated by NF-κB signaling than others. Within each peak, the precise location where NF-κB binding is most enriched is found at the summit points of the peaks. Similar to peaks, summit regions are found in various locations across all chromosomes (FIG. 8B). The summits provide information to further characterize specific gene regions involved in the NF-κB signaling pathways.

### Example 9. Genetic Reads Match Expected NF-κB Profile

To confirm that the observed GWAS data matches the NF-κB profile, the distribution of fragment size, peak width, read mapping, and peak-transcription start site distance were examined (FIG. 4). At the genome-wide level, it was found that Rela/p65 bound to sequences with an average size of 336 bp, and there were a total of 9944 peaks and 10568 summits (FIG. 4A). The abundant binding frequencies match the known role of NF-κB as a critical regulator of gene expression across the genome. 16 Peak width represents the length of the DNA bound by Rela/p65, and it was shown that most of the peak areas had around 500 bases (FIG. 4B). This peak width range is similar to that observed in previous studies.¹⁷

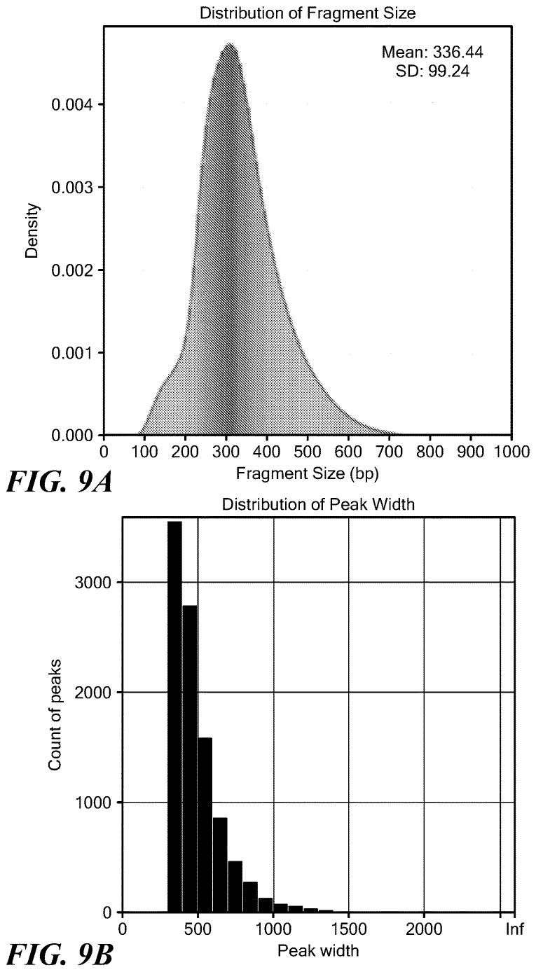

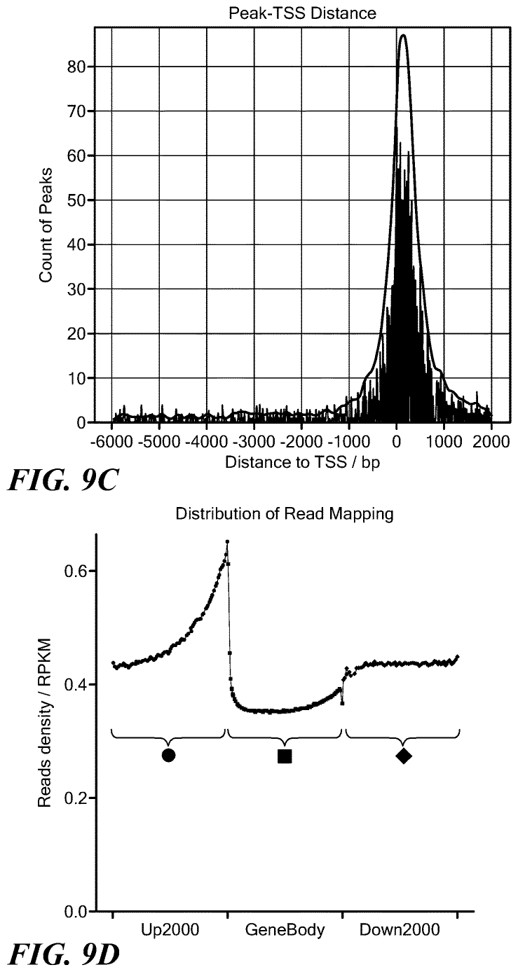

To determine the exact position of NF-κB on a specific gene, proximity to transcription start sites (TSS) was tested. A TSS is the location of the first nucleotide that RNA polymerase binds to for initiation of gene transcription, and the proximity of regulatory elements to the TSS can significantly impact the rate and level of gene expression. In the present analysis, regions of 2 kb upstream and downstream of the gene were divided into 100 equal parts, and the distribution of read density (mapped reads/total reads) was plotted. The plots in FIGS. 9A-9B show that peak counts were distributed across the 2 kb upstream and downstream of the gene region. However, most of the peaks were observed near the TSS “0”, indicating that NF-κB binds mostly at the location where transcription starts (FIG. 9C). Also analyzed was the mapping position distribution of the ratio of mapped reads to total reads and it was found that most of the read density is located within 2 kb upstream of the gene body, with the highest density around the start of the gene body, corresponding to the transcription site binding by Rela/p65 (FIG. 9D). These findings match the expected profile of a transcription factor such as NF-κB, which binds near the start of gene sequencing to regulate its expression.¹⁸ Taken together, these results show a pattern closely aligned with expectations for NF-κB, validating the droplet CUT&MAP approach and accurate genome-wide mapping of NF-κB and other transcription factors.

### Example 10. Gene Interactions Display Complex Cellular Response

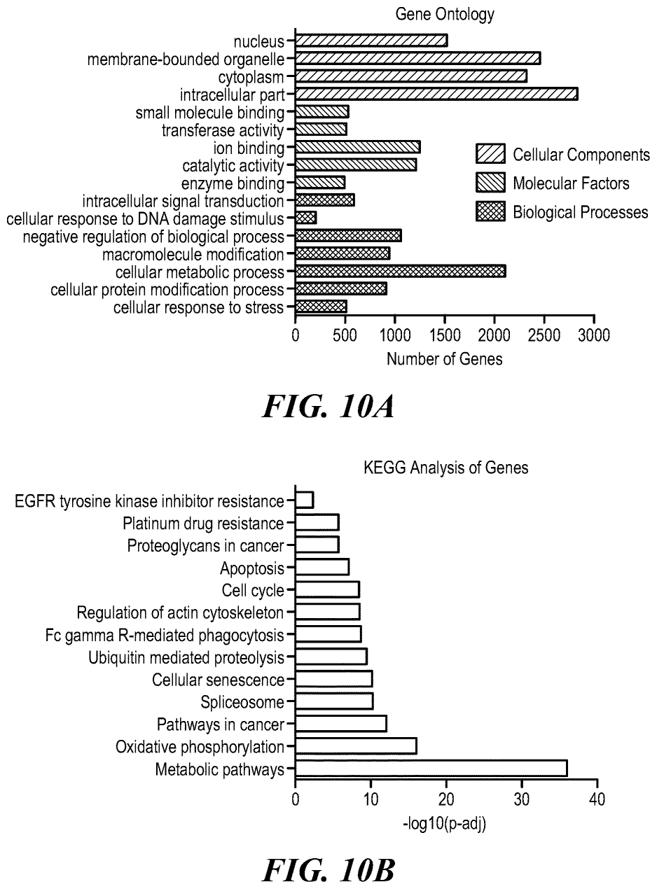

Gene Ontology (GO) and Kyoto Encyclopedia of Genes and Genomes (KEGG) biological process enrichment analysis were performed to further characterize the pathways regulated by NF-κB. It was found that cellular metabolism and protein modifications were among the top biological processes associated with NF-κB binding sites (FIG. 10A). It was observed that processes involved in protein catabolismand proteolysis were enriched, suggesting possible cytotoxic susceptibility mechanisms. Notably, NF-κB also has both regulatory and activating roles in protein and enzyme binding. Furthermore, there was a high number of genes related to cellular components throughout the cell, including organelles, the nucleus, and the cytoplasm. The findings show the vast range of potential impacts of NF-κB activity.

Looking at the KEGG terms, several hits related to cancer activities were found, including pathways in cancer and platinum drug resistance (FIG. 10B). Conversely, there were several terms related to potential cell inactivity, including apoptosis and cellular senescence. Observing the overall influence of NF-κB on cell growth or cell death is essential in determining whether it is a treatable pathway for specific cancers. For K562 killed by NK92, a largely split profile was observed towards both potential apoptosis-inducing and resistance-inducing pathways. The high gene number observed in metabolic pathways and the high rich factor observed for oxidative phosphorylation indicate a trend towards a resistant outcome, matching the known response to NF-κB for K562.¹⁹

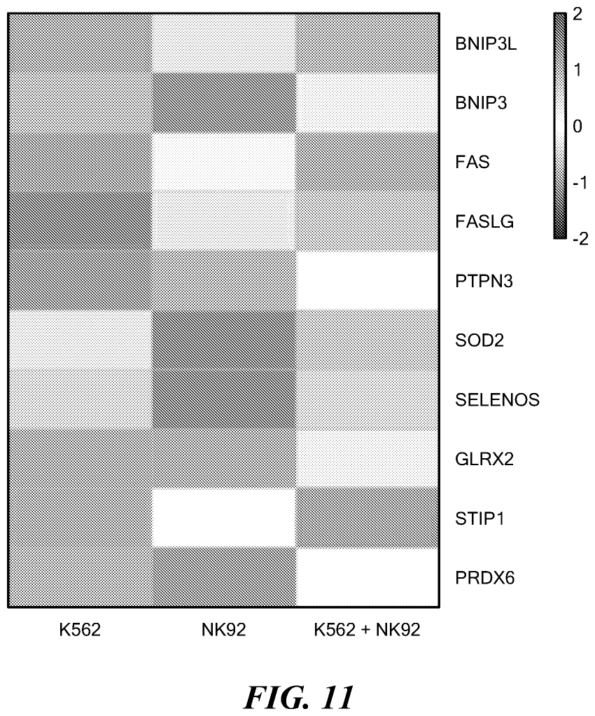

To take a closer look at the genes that were influenced by Rel-A binding in these K562 cells, the gene list under the enriched GO terms was further examined (Table 1). Several hits were found matching previous observations in the literature, both promoting resistance and potentially apoptosis in the K562 cells. Some of the gene results have not been frequently cited for responding to NF-κB activity, indicating potential interactions of NF-κB with genes that may be specific to the K562 cellular response to immune cells. To verify that this NF-κB activity was related to the K562 response to NK92, transcriptomic sequencing also was performed of K562 and NK92 cells alone and co-encapsulated in droplets (FIG. 11). Reduced expression was observed of BNIP3 mRNA in co-encapsulated K562 and increased FAS and SOD2 expression compared to K562 cultured alone. These findings correlate well with the expected influence of NF-κB activity, which has been shown to inhibit transcription of BNIP3 but increase FAS and SOD2 expression.^{20,21 22} The results demonstrate that the NF-κB binding profile observed through the Iso CUT&MAP droplet assay is representative of the K562 cell response to NK92 cells.

TABLE 1
List detailing some of the genes found in association with Rel-A from the
Iso CUT&MAP droplet assay potentially related to K562 survival. Expected
influence on cell survival and function of gene expression is based on
the literature related to these genes and their NF-kB interactions.

| | | | | Function |
|---|---|---|---|---|
| | | Apoptosis or | | Previously |
| | | Resistance | | Associated |
| Gene ID | Gene Name | Inducing | Function | with NFkB |
| BNIP3 | BCL2 interacting | Anti-apoptotic | BNIP3 activations induces | Yes |
| | protein 3 | | apoptosis through the Bcl-2 | |
| | | | protein | |
| FAS | Fas cell surface | Pro-apoptotic | Binding of Fas by FasL | Yes |
| | death | | induces apoptosis in cells. | |
| | | | Upregulated by NF- [symbol] | |
| PTPN3 | Protein tyrosine | Anti-apoptotic | Inhibits Fas-mediated apoptosis | Yes |
| | phosphatase non- | | signal transduction, promoting | |
| | receptor type 13 | | cell survival._{25, 26} | |
| SOD2 | Superoxide | Pro-resistance | Antioxidant essential for cell | Yes |
| | dismutase 2 | | survival against oxidative | |
| | | | stress.₂₀ | |
| SELENOS | Selenoprotein | Pro-resistance | Produces various enzymes, | Yes |
| | | | contributing to drug and stress | |
| | | | resistance.27 | |
| GLRX2 | Glutaredoxin 2 | Pro-resistance | Protects against oxidation, | No |
| | | | attenuates NF-kB signaling.28 | |
| STIP1 | Stress induced | Pro-resistance | Promotes cell migration and | No |
| | phosphoprot [symbol] | | cancer cell metastasis.29 | |
| PRDX6 | Peroxiredoxin | Anti-resistance | Antioxidant. Expression found | No |
| | | | to reduce tumor cell metastasis | |
| | | | and [symbol] | |

[symbol]

indicates data missing or illegible when filed

Most genes found in these tests correspond to cellular resistance. However, other genes like FAS and PRDX6 are expected to make NK cells more susceptible to killing. This information can be valuable when tailoring cancer therapies, as inhibiting NF-κB activity may reduce immune cell killing through these pathways. In cells that respond favorably to NF-κB activity and may be sensitized by promoting NF-κB, this type of data can help identify targets to improve treatment efficacy. In this particular study, both Fas and Fas ligands showed increased expression in the co-encapsulated cells, which is a potent pathway for immune cell mediated cytotoxicity of cancer cells.²³ Inhibiting NF-κB with a TNFα inhibitor, as has been proposed in some studies, may result in the decreased killing of cancer cells in this instance, regardless of other resistance pathways also being suppressed. In the present study, genes associated with Rel-A were observed that have not been well documented in NF-κB signaling, including GLRX2, STIP1, and PRDX6. These genes play a role in K562 cell response to immune cells. They also display the ability of the Iso CUT&MAP method to reveal new genes affecting cancer cell resistance in response to transcription factor activity.

### Example 11. Analysis of Transcriptional Regulation by DNA Hypermethylation

Transcription factors directly control the transcription of key regulators in major signaling pathways. Dysregulation in transcription factors or their binding sites plays a significant role in cancer pathogenesis. It is recognized that in various types of cancer, DNA hypermethylation within the promoter region of specific tumor-suppressor genes leads to gene silencing and subsequent reduction of transcript and protein expressions of those genes. As a result, the interplay between DNA-protein interactions and epigenetic modifications contribute to the heterogeneous array of phenotypes and behaviors exhibited by tumor cells.

The present technology is used in multiplex format to capture 1) both transcription factor-bound DNA and total mRNA for the characterization of DNA-protein interactions, and 2) methylated DNA and total mRNA for characterization of transcriptional regulation.

### 1. Multiplex Antibody-Conjugated Magnetic Bead System for DNA-Protein Interactions

Transcription factor protein-bound magnetic beads are used to capture and sequence DNA bound to selected transcription factors in tumor cells. Subsequently, mapping of transcription factor binding sites across the genome is carried out. At the same time, transcriptome profiles are generated from the same tumor cells using magnetic beads that capture total mRNA, and distinctions are explored in the biological processes, signature pathways, and molecular functions between tumor cells that are sensitive and resistant to immune cell-induced killing events.

### 2. Simultaneous Characterization of Epigenetic Modification and Transcriptomic Profile of Tumor Cells

The impact of CpG island hypermethylation on gene inactivation in tumor cells is analyzed using multiplexing of magnetic beads in the co-encapsulation of tumor and effector cells to capture methylated DNA and total mRNA simultaneously from the same droplet after incubation in the droplet array. After acquiring the nucleic acid samples, sequencing and analysis are performed to identify the distribution of methylated gene sites across the genome and their corresponding levels of transcript expression. By conducting comparative analysis between the transcriptomic and epigenetic profiles of the killed versus non-killed tumor cells, key differentially expressed genes and pathways are identified that drive tumor progression under epigenetic regulation.

### Example 12. Isolation and Sequencing of mRNA, Methylated RNA, and Methylated DNA

When mRNA molecules undergo methylation, this can alter their stability and interactions, impacting overall gene expression levels. The abundance of m6A within mRNA transcripts has been shown to affect RNA metabolism, encompassing aspects like translation capacity, stability, and the transcription of RNA. Similarly, transcription factors are pivotal in governing gene expression and are central to diverse biological processes such as immune response, cellular differentiation, and homeostasis. Transcription factors also possess the ability to exert influence over the epigenetic configuration of specific regions within the genome.

1. Optimization of mRNA Isolation and Sequencing from Killed Vs. Not Killed Tumor Cells

Target tumor cells are co-encapsulated pairwise with NK92 immune effector cells and magnetic mRNA Dynabeads Oligo (dT) 25 (Cat #61002) in droplets. After selective release and collection, droplets containing dead tumor cells and mRNA beads are directly placed on a 0.5-0.7 mL magnetic rack tube to isolate beads containing mRNA material. The non-killed tumor cells require manual lysis outside the microfluidic device. The mRNA Dynabeads capture the immune-mediated cytotoxicity-resistant tumor cells' mRNA material. Once the mRNA beads containing mRNA material from both tumor cell types are isolated, a washing buffer is used to elute the mRNA from the magnetic beads. RNA sequencing is then performed on sensitive and resistant tumor samples to reveal transcriptome elements or genes relevant to tumor behavior.

### 2. Antibody-Conjugated Magnetic Bead System for Targeted Transcription Factor-Bound DNA Pull Down

Carboxylated magnetic beads covalently coupled with antibodies against a specific transcription factor are used to capture DNA fragments that interact and bind with the transcription factor. DNA fragments within the dead tumor cells are exposed, allowing antibody-conjugated beads to attach at their associated binding sites. However, the immune-mediated cytotoxicity-resistant tumor cells retain their intact cellular structures, requiring manual lysis outside the microfluidic device. Once DNA fragments from both groups (killed and non-killed tumor cells) are captured, they are subjected to sequencing for genome-wide transcription factor binding site mapping.

### 3. Isolation and Sequencing of Methylated RNA and DNA Using Conjugated Magnetic Capture Beads

The quantification of global levels of methylated RNA can be achieved by utilizing antibodies with a high affinity for specific RNA methylation markers like N6-methyladenosine (m6A). Methylated RNA Immunoprecipitation sequencing (MeRIP-seq) is used to effectively chart the distribution of methylated RNA markers (m6A) within purified mRNA transcripts. mRNA is isolated from both the killing and non-killing interactions of tumor and effector cells. For the dead tumor cells, mRNA Dynabeads are isolated from droplets and purified for downstream methylation sequencing. The non-killed tumor cells are separated from the NK cells using a spin column and subjected to manual lysis to expose RNA fragments in the nucleus. The purified mRNA samples are then fragmented into segments spanning about 70 to 100 nucleotides before immunoprecipitation. The isolated (fragmented) mRNA is combined with antibody-conjugated magnetic beads such as m6A antibody-coupled magnetic Dynabeads. The RNA fragments harboring m6A modifications are then released from the antibody-bound beads and subjected to an additional round of immunoprecipitation to ensure a high resolution of RNA containing m6A methylomes. The resultant pool of RNA, enriched with m6A, is then subjected to RNA sequencing (RNA-seq) analysis.

For DNA methylation, methylated DNA immunoprecipitation sequencing (MeDIP-Seq) is employed, which is often used to study DNA methylation markers such as 5-hydroxymethylcytosine (5-hmC) and 5-methylcytosine (5 mC). Similar to the process described for RNA methylation isolation, purified DNA isolated from killed and non-killed tumor cells is subjected to immunoprecipitation to isolate methylated DNA. Non-transcription factor bound DNA magnetic beads are incorporated into droplet generation with tumor and effector cells. After 6 hours of incubation on the droplet array, killed and non-killed tumor cells are selectively released on the microvalve device. For the dead tumor cells, DNA-bound beads are isolated and purified for downstream processes. The non-killed tumor cells are separated from the NK cells using a spin column and subjected to manual lysis to expose DNA fragments in the nucleus. From there, the same DNA magnetic beads are implemented to isolate DNA material from the non-killed tumor population. Antibody-conjugated magnetic beads specific to methylated DNA are then added to purified DNA samples and precipitated to provide a high resolution of DNA containing 5-hmC or 5-mC specific methylomes. The methylated DNA is then subjected to DNA sequencing.

4. Isolation of mRNA from mRNA Specific Magnetic Dynabeads.

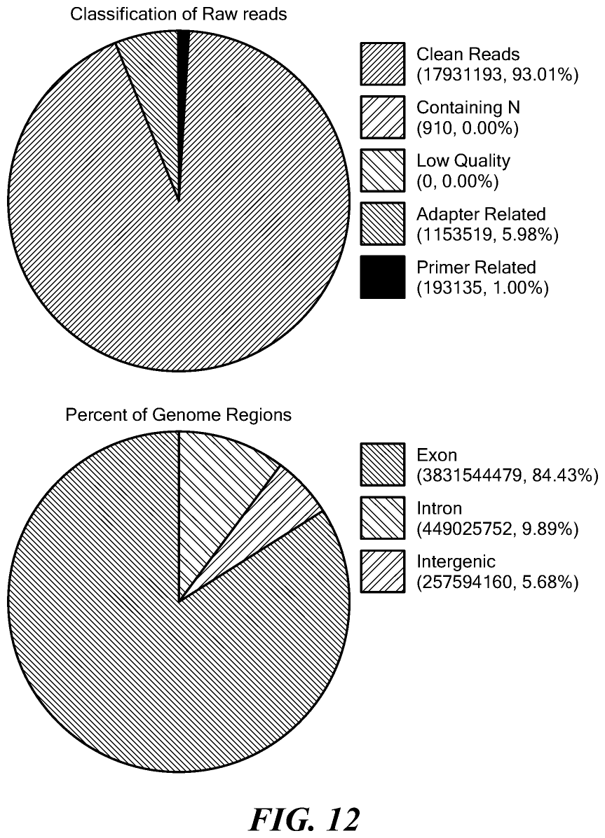

One million K562 cells, 10 μl magnetic mRNA Dynabeads Oligo (dT) 25 Cat #61002, and three million NK92 cells were encapsulated within droplets in a microfluidic device. Droplets with both cell samples and beads were collected and incubated in an Eppendorf tube for 6 hours at 37 C. The resulting data shows the percentage of raw reads free of adapter contamination and void of low-quality nucleotides. The data also revealed that the mapped regions are more abundant in the exon region of the genome (FIG. 12). Altogether, this indicates that the mRNA extracted is intact and not heavily degraded or contaminated.

### REFERENCES

Agnihotri, S. N., Ugolini, G. S., Sullivan, M. R., Yang, Y., Ganzo, A. D., Lim, J. W., & Konry, T. Droplet microfluidics for functional temporal analysis and cell recovery on demand using microvalves: Application in immunotherapies for cancer. Lab on a Chip (2022) 22, 3258, doi.org/10.1039/D2LC00435F
He, B., Lu, N., & Zhou, Z. (2009). Cellular and Nuclear Degradation during Apoptosis. Current Opinion in Cell Biology, 21 (6), 900-912. doi.org/10.1016/j.ceb.2009.08.008
Hu, X., Li, Z., Lin, R., Shan, J., Yu, Q., Wang, R., Liao, L., Yan, W., Wang, Z., Shang, L., Huang, Y., Zhang, Q., & Xiong, K. (2021). Guidelines for Regulated Cell Death Assays: A Systematic Summary, A Categorical Comparison, A Prospective. Frontiers in Cell and Developmental Biology, 9. www.frontiersin.org/articles/10.3389/fcell.2021.634690
Janssens, D., & Henikoff, S. (2019 May 9). CUT&RUN: Targeted in situ genome-wide profiling with high efficiency for low cell numbers. Protocols.lo. www.protocols.io/view/cut-amp-run-targeted-in-situ-genome-wide-profiling-zcpf2vn
Jordan Rowley, M., & Corces, V. G. (2018). Organizational Principles of 3D Genome Architecture. Nature Reviews. Genetics, 19 (12), 789-800. doi.org/10.1038/s41576-018-0060-8
Majtnerová, P., & Roušar, T. (2018). An overview of apoptosis assays detecting DNA fragmentation. Molecular Biology Reports, 45 (5), 1469-1478. doi.org/10.1007/s11033-018-4258-9
Meers, M. P., Tenenbaum, D., & Henikoff, S. (2019). Peak calling by Sparse Enrichment Analysis for CUT&RUN chromatin profiling. Epigenetics & Chromatin, 12, 42. doi.org/10.1186/s13072-019-0287-4
Mehta, M., Raguraman, R., Ramesh, R. & Munshi, A. (2022). RNA binding proteins (RBPs) and their role in DNA damage and radiation response in cancer. Advanced Drug Delivery Reviews, 191, 114569. doi.org/10.1016/j.addr.2022.114569.
Ramachandran, S., Ahmad, K., & Henikoff, S. (2017). Transcription and remodeling produce asymmetrically unwrapped nucleosomal intermediates. Molecular Cell, 68 (6), 1038-1053.e4. doi.org/10.1016/j.molcel.2017.11.015
Skene, P. J., & Henikoff, S. (2017). An efficient targeted nuclease strategy for high-resolution mapping of DNA binding sites. ELife, 6, e21856. doi.org/10.7554/eLife.21856
Yu, F., Sankaran, V. G., & Yuan, G.-C. (2022). CUT&RUNTools 2.0: A pipeline for single-cell and bulk-level CUT&RUN and CUT&Tag data analysis. Bioinformatics, 38 (1), 252-254. doi.org/10.1093/bioinformatics/btab507
(1) Singh, H.; Khan, A. A.; Dinner, A. R. Gene regulatory networks in the immune system. Trends Immunol 2014, 35 (5), 211.
(2) Lambert, S. A.; Jolma, A.; Campitelli, L. F.; Das, P. K.; Yin, Y.; Albu, M.; Chen, X.; Taipale, J.; Hughes, T. R.; Weirauch, M. T. The Human Transcription Factors. Cell 2018, 172 (4), 650.
(3) Nakato, R.; Shirahige, K. Recent advances in ChIP-seq analysis: from quality management to whole-genome annotation. Brief Bioinform 2017, 18 (2), 279.
(4) Nakato, R.; Sakata, T. Methods for ChIP-seq analysis: A practical workflow and advanced applications. Methods 2021, 187, 44.
(5) Agnihotri, S. N.; Ugolini, G. S.; Sullivan, M. R.; Yang, Y.; De Ganzo, A.; Lim, J. W.; Konry, T. Droplet microfluidics for functional temporal analysis and cell recovery on demand using microvalves: application in immunotherapies for cancer. Lab Chip 2022, 22 (17), 3258.
(6) Giridharan, S.; Srinivasan, M. Mechanisms of NF-kappaB p65 and strategies for therapeutic manipulation. J Inflamm Res 2018, 11, 407.
(7) Sarkar, S.; Motwani, V.; Sabhachandani, P.; Cohen, N.; Konry, T. T Cell Dynamic Activation and Functional Analysis in Nanoliter Droplet Microarray. J Clin Cell Immunol 2015, 6 (3).
(8) Sarkar, S.; Cohen, N.; Sabhachandani, P.; Konry, T. Phenotypic drug profiling in droplet microfluidics for better targeting of drug-resistant tumors. Lab Chip 2015, 15 (23), 4441.
(9) Sarkar S, S. P., Stroopinsky D, Palmer K, Cohen N, Rosenblatt J, Avigan D, Konry T. Dynamic analysis of immune and cancer cell interactions at single cell level in microfluidic droplets. Biomicrofluidics 2016, 10 (5): 054115.
(10) Zhang, Y.; Chen, X.; Gueydan, C.; Han, J. Plasma membrane changes during programmed cell deaths. Cell Res 2018, 28 (1), 9.
(11) Gadiyar, V.; Lahey, K. C.; Calianese, D.; Devoe, C.; Mehta, D.; Bono, K.; Desind, S.; Davra, V.; Birge, R. B. Cell Death in the Tumor Microenvironment: Implications for Cancer Immunotherapy. Cells 2020, 9 (10).
(12) Lindenboim, L.; Zohar, H.; Worman, H. J.; Stein, R. The nuclear envelope: target and mediator of the apoptotic process. Cell Death Discov 2020, 6, 29.
(13) Elmore, S. Apoptosis: a review of programmed cell death. Toxicol Pathol 2007, 35 (4), 495.
(14) Gauthier, B. R.; Comails, V. Nuclear Envelope Integrity in Health and Disease: Consequences on Genome Instability and Inflammation. Int J Mol Sci 2021, 22 (14).
(15) Park MH, H. J. Roles of NF-κB in Cancer and Inflammatory Diseases and Their Therapeutic Approaches. Cells 2016, 5 (2): 15.
(16) Brignall, R.; Moody, A. T.; Mathew, S.; Gaudet, S. Considering Abundance, Affinity, and Binding Site Availability in the NF-kappaB Target Selection Puzzle. Front Immunol 2019, 10, 609.
(17) Ngo, K. A.; Kishimoto, K.; Davis-Turak, J.; Pimplaskar, A.; Cheng, Z.; Spreafico, R.; Chen, E. Y.; Tam, A.; Ghosh, G.; Mitchell, S.et al. Dissecting the Regulatory Strategies of NF-kappaB RelA Target Genes in the Inflammatory Response Reveals Differential Transactivation Logics. Cell Rep 2020, 30 (8), 2758.
(18) Georgakopoulos-Soares, I.; Deng, C.; Agarwal, V.; Chan, C. S. Y.; Zhao, J.; Inoue, F.; Ahituv, N. Transcription factor binding site orientation and order are major drivers of gene regulatory activity. Nat Commun 2023, 14 (1), 2333.
(19) Morotti A, C. D., Pautasso M, Messa F, Arruga F, Defilippi I, Carturan S, Catalano R, Rosso V, Chiarenza A, Taulli R, Bracco E, Rege-Cambrin G, Gottardi E, Saglio G. NF-κB inhibition as a strategy to enhance etoposide-induced apoptosis in K562 cell line . . . 2006, 81 (12): 938-45.
(20) Xu, Y.; Fang, F.; Dhar, S. K.; St Clair, W. H.; Kasarskis, E. J.; St Clair, D. K. The role of a single-stranded nucleotide loop in transcriptional regulation of the human sod2 gene. J Biol Chem 2007, 282 (22), 15981.
(21) Singh, N. P.; Nagarkatti, M.; Nagarkatti, P. S. Role of dioxin response element and nuclear factor-kappaB motifs in 2,3,7,8-tetrachlorodibenzo-p-dioxin-mediated regulation of Fas and Fas ligand expression. Mol Pharmacol 2007, 71 (1), 145.
(22) Shaw, J.; Zhang, T.; Rzeszutek, M.; Yurkova, N.; Baetz, D.; Davie, J. R.; Kirshenbaum, L. A. Transcriptional silencing of the death gene BNIP3 by cooperative action of NF-kappaB and histone deacetylase 1 in ventricular myocytes. Circ Res 2006, 99 (12), 1347.
(23) Peter, M. E.; Hadji, A.; Murmann, A. E.; Brockway, S.; Putzbach, W.; Pattanayak, A.; Ceppi, P. The role of CD95 and CD95 ligand in cancer. Cell Death Differ 2015, 22 (4), 549.
(24) Baetz, D.; Regula, K. M.; Ens, K.; Shaw, J.; Kothari, S.; Yurkova, N.; Kirshenbaum, L. A. Nuclear factor-kappaB-mediated cell survival involves transcriptional silencing of the mitochondrial death gene BNIP3 in ventricular myocytes. Circulation 2005, 112 (24), 3777.
(25) Irie, S.; Li, Y.; Kanki, H.; Ohyama, T.; Deaven, L. L.; Somlo, S.; Sato, T. A. Identification of two Fas-associated phosphatase-1 (FAP-1) promoters in human cancer cells. DNA Seq 2001, 11 (6), 519
(26) Ivanov, V. N.; Ronai, Z.; Hei, T. K. Opposite roles of FAP-1 and dynamin in the regulation of Fas (CD95) translocation to the cell surface and susceptibility to Fas ligand-mediated apoptosis. J Biol Chem 2006, 281 (3), 1840.
(27) Gao, Y.; Hannan, N. R.; Wanyonyi, S.; Konstantopolous, N.; Pagnon, J.; Feng, H. C.; Jowett, J. B.; Kim, K. H.; Walder, K.; Collier, G. R. Activation of the selenoprotein SEPS1 gene expression by pro-inflammatory cytokines in HepG2 cells. Cytokine 2006, 33 (5), 246.
(28) Liao, B. C.; Hsieh, C. W.; Lin, Y. C.; Wung, B. S. The glutaredoxin/glutathione system modulates NF-kappaB activity by glutathionylation of p65 in cinnamaldehyde-treated endothelial cells. Toxicol Sci 2010, 116 (1), 151.
(29) Beckley S J, H. M., Kituyi S N, Wingate I, Chakraborty A, Schwarz K, Makhubu M P, Rousseau R P, Ruck D K, de la Mare J A, Blatch G L, Edkins A L. STIP1/HOP Regulates the Actin Cytoskeleton through Interactions with Actin and Changes in Actin-Binding Proteins Cofilin and Profilin. Int J Mol Sci 2020, 21 (9): 3152.
(30) Min, Y.; Kim, M. J.; Lee, S.; Chun, E.; Lee, K. Y. Inhibition of TRAF6 ubiquitin-ligase activity by PRDX1 leads to inhibition of NFkB activation and autophagy activation. Autophagy 2018, 14 (8), 1347.

1. A method for epigenetic analysis of one or more cytotoxic immune cells, the method comprising the steps of:
(a) providing a first aqueous suspension comprising cytotoxic immune cells, a second aqueous suspension comprising target cells, and a microfluidic device configured for co-encapsulating and imaging individual said cytotoxic immune cells with individual said target cells in aqueous microdroplets in an oil stream;
(b) forming a plurality of said aqueous microdroplets, distributing and imaging the microdroplets in a plurality of docking stations in said microfluidic device, and incubating and imaging the microdroplets for a period of time;
(c) selecting a portion of the microdroplets in which killing of a said target cell has occurred according to selected criteria;
(d) collecting and merging the selected microdroplets;
(e) fixing the contents of the merged microdroplets with a chemical fixing agent, whereby cytotoxic immune cells are fixed;
(f) lysing the fixed cells and isolating nuclei of the fixed cytotoxic immune cells;
(g) immobilizing the isolated nuclei on a solid support;
(h) incubating the immobilized nuclei with an antibody that specifically binds to a selected nucleic acid-binding protein;
(i) incubating the immobilized nuclei with a nuclease, whereby nucleic acid not bound to the selected nucleic acid-binding protein is degraded, leaving nucleic acid fragments that were bound to the selected nucleic acid-binding protein;
(j) isolating said nucleic acid fragments; and
(k) sequencing one or more of the isolated nucleic acid fragments, whereby one or more nucleic acids interacting with the selected nucleic acid-binding protein in said one or more cytotoxic immune cells are identified.

2. The method of claim 1, wherein the one or more cytotoxic immune cells comprise NK cells.

3. The method of claim 1, wherein the target cells comprise tumor cells or cells infected with a microbial pathogen.

4. The method of claim 1, wherein the incubation period of time is sufficient to allow killing of at least a portion of the target cells by the co-encapsulated cytotoxic immune cells.

5. The method of claim 1, wherein the selected criteria of step (c) comprise elapsed time to killing of a target cell.

6. The method of claim 1, wherein the selected nucleic acid-binding protein is a transcription factor.

7. The method of claim 1, wherein the nuclease is micrococcal nuclease.

8. The method of claim 1, wherein step (j) further comprises amplifying the nucleic acid fragments, such as by polymerase chain reaction or rolling circle amplification.

9. The method of claim 8, wherein the epigenetic analysis is performed on a single cytotoxic immune cell.

10. The method of claim 1, wherein step (j) further comprises preparing a library of said nucleic acid fragments.

11. (canceled)

12. A method of epigenetic analysis of one or more target cells following interaction between said one or more target cells and one or more cytotoxic immune cells, the method comprising:
(a) providing a first aqueous suspension comprising cytotoxic immune cells, a second aqueous suspension comprising target cells and microbeads coated with a capture agent for a selected target cell nucleic acid-binding protein, and a microfluidic device configured for co-encapsulating and imaging individual said cytotoxic immune cells with individual said target cells in aqueous microdroplets in an oil stream;
(b) forming a plurality of said aqueous microdroplets, distributing and imaging the microdroplets in a plurality of docking stations in said microfluidic device, and incubating and imaging the microdroplets for a period of time, whereby in a portion of the microdroplets a said target cell has been killed by a said cytotoxic immune cell, and whereby the microbeads bind to the nucleic acid-binding protein in the killed target cells;
(c) selecting a portion of the microdroplets in which killing of a said target cell has occurred according to selected criteria;
(d) collecting and merging the selected microdroplets;
(e) isolating said microbeads;
(f) releasing from the isolated microbeads nucleic acid fragments that were bound to said nucleic acid-binding protein;
(g) isolating said nucleic acid fragments; and
(h) sequencing one or more of the isolated nucleic acid fragments, whereby one or more nucleic acids interacting with the nucleic acid-binding protein in said one or more target cells are identified.

13. The method of claim 12, wherein the capture agent is an antibody or an aptamer.

14. The method of claim 12, wherein the one or more cytotoxic immune cells comprise NK cells.

15. The method of claim 12, wherein the target cells comprise tumor cells or cells infected with a microbial pathogen.

16. The method of claim 12, wherein the incubation period of time is sufficient to allow killing of at least a portion of the target cells by the co-encapsulated cytotoxic immune cells.

17. The method of claim 12, wherein the selected criteria of step (c) comprise elapsed time to killing of a target cell.

18. The method of claim 12, wherein the selected nucleic acid-binding protein is a transcription factor.

19. The method of claim 12, wherein step (g) further comprises amplifying the nucleic acid fragments, such as by polymerase chain reaction or rolling circle amplification.

20. The method of claim 19, wherein the epigenetic analysis is performed on a single target cell.

21. The method of claim 12, wherein step (g) further comprises preparing a library of said nucleic acid fragments.

22. (canceled)

23. The method of claim 12, wherein the microbeads are magnetic, and a magnet is used to isolate the microbeads in step (e).

24. The method of claim 12, wherein the cytotoxic immune cells are NK cells and the target cells are tumor cells or cells infected with a microbial pathogen, and both types of cells are obtained from the same subject.

25. The method of claim 12, wherein two or more different antibodies that bind to two or more different nucleic acid binding proteins, or two or more different types of microbeads, each type of microbeads having a different said capture agent, are used, and the method is carried out in a multiplex format.

26. A kit for epigenetic analysis of a cytotoxic immune cell, the kit comprising:
(i) a microfluidic device configured for co-encapsulating and imaging individual said cytotoxic immune cells with individual said target cells in aqueous microdroplets in an oil stream; and
(ii) an antibody that specifically binds a selected nucleic acid binding protein of the cytotoxic immune cell.

27. (canceled)

28. A kit for epigenetic analysis of a target cell, the kit comprising:
(i) a microfluidic device configured for co-encapsulating and imaging individual said cytotoxic immune cells with individual said target cells in aqueous microdroplets in an oil stream; and
(ii) a plurality of microbeads comprising a capture agent, such as an antibody or aptamer, that specifically binds a selected nucleic acid binding protein of the target cell.

29. (canceled)
